(19) United States
(12) Patent Application Publication
(10) Pub. No.: US 2007/0191773 A1
(43) Pub. Date: Aug. 16, 2007

(54) Low profile infusion set

(75) Inventor: Steven E. Wojcik, Shoreline, WA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Animas Corporation

(21) Appl. No.: 11/355,780

(22) Filed: Feb. 16, 2006

(51) Int. Cl. A61M 5/178

(52) U.S. Cl. 604/158; 604/93.01

(57) ABSTRACT

An infusion set comprising a cannula housing portion having a base surface and a septum housing portion pivotally attached to said cannula housing portion. The septum housing portion having an axis and being pivotable between a first position wherein the axis is substantially perpendicular to the base surface and a second position wherein the axis is substantially parallel to the base surface. A cannula is supported in and extends from the septum housing portion such that the cannula is substantially parallel to the axis when the septum housing portion is in the first position.

### FIELD OF THE INVENTION

The present invention relates generally to an infusion set, and more particularly to a low profile infusion set used for intermittent or continuous delivery of medication, such as insulin to a patient.

### BACKGROUND OF THE INVENTION

Patients who receive intermittent or continuous doses of medication, such as insulin, via subcutaneous injection, often have an infusion set affixed to their skin in a convenient location. Keeping an infusion set fixed in place is discreet, and reduces the need for repeatedly puncturing the skin with a needle, thereby reducing the risk of infection as well as reducing the formation of scar tissue. The infusion set typically includes a housing supporting a tubular cannula with a removable injection needle at one end for penetrating the skin, and a septum at the other end for receiving a needle attached to a supply tube from a medicinal source, e.g., an insulin pump. One well-known conventional infusion set is a “straight set”, in which the cannula and injection needle are inserted in an orientation substantially normal to the skin. The straight set requires a relatively short injection needle, which is less intimidating to some patients, and is relatively easy to insert through the skin. But, because the cannula and injection needle are supported to be oriented normal to the skin, the housing must be upright, conspicuous, and relatively bulky, and furthermore, the cannula, rigidly attached to a bottom of the housing can be subject to kinking and occlusion.

Another known infusion set is a low profile angled set, in which the cannula and injection needle are supported in the housing to be oriented at an acute angle with respect to the skin. The housing of the low profile angled set is less bulky and is much more discreet than the housing of the straight set. However, because of the angled insertion, a much longer injection needle is required, and the longer needle is more intimidating and more difficult to insert, and is subject to inadvertent bending.

Additional problems exist with both the conventional straight and angled sets. For example, a relatively long portion of cannula tubing is left exposed. A view of the injection site is often obscured. Adhesive mounting pads used on the sets can be awkward to use, often prematurely contacting the skin, causing wrinkling of the adhesive pad. Moreover, the needle or the cannula often touch non-sterile tissue or clothing prior to insertion, which increases the risk of infection.

It is desirable, therefore, to provide a low profile, compact infusion set with a relatively short needle, which is easy to position and insert, is easy to connect to the medical source, which does not obscure the patient's view of the injection site, is low and discreet, and in which the cannula is protected from kinking and occlusion.

### SUMMARY OF THE INVENTION

Accordingly, the present invention is directed to an infusion set that mitigates or substantially obviates one or more of the shortcomings caused by the limitations and disadvantages of the related art.

The features and advantages of the invention will be set forth in the description which follows, and in part will be apparent from the description, or may be learned by practice of the invention. The advantages of the invention will be realized and attained by the apparatus, and the method of practicing the invention, particularly pointed out in the written description and claims below, as well as in the attached drawings.

To achieve the advantages of the invention, an infusion set is provided with a multiple-part housing. A first or cannula housing portion is removably attachable to a surface of a user's skin. A second or septum housing portion is pivotally attached to the cannula housing portion, pivotable between a first position above the cannula housing portion and substantially normal to the surface of the skin and a second position alongside the cannula housing portion and substantially parallel to the skin surface. An elongated tubular cannula is provided, having a first end, first and a second intermediate portions, and a second end. An injection needle can be removably mounted in the first end. The first intermediate portion is supported in the cannula housing portion, and is oriented so that the injection needle and the first end of the cannula penetrate the skin surface at an injection site in an orientation that is substantially normal to the skin surface. A septum is inserted in the second end of the cannula, and the cannula second end, cannula second intermediate portion and the septum are supported in the septum housing portion.

In accordance with an aspect of the invention, a cannula guide or mandrel is also provided in the cannula housing portion, which imposes a turn in the cannula first intermediate portion proximate the skin surface. This turn is desirably a fraction of a 90° turn, and this turn prevents the cannula from kinking or being pinched and occluded.

In accordance with another aspect of the invention, an adhesive assembly is provided on an exterior bottom of the cannula housing portion for adhering the cannula housing portion to the skin. The adhesive assembly includes an adhesive surface and a paper backing. Desirably, the adhesive pad has a center portion and side portions, and the paper backing is split into two or more portions that are separately and sequentially removable from the adhesive pad.

In accordance with a further aspect of the invention, the adhesive assembly defines a base for the cannula housing portion, and has an aperture, and the injection needle and the first end of the cannula pass through this aperture to penetrate the user's skin. The first intermediate portion of the cannula is protected inside the cannula housing portion and the injection site is shielded by the housing from inadvertent contact with the user's skin or clothing. An optional annular shield proximate the aperture shields the injection site from contamination, while still allowing the user to view the infusion site from above.

In accordance with yet another aspect of the invention, the point of pivotal attachment between the septum housing portion and the cannula housing portion is preselected so that, after insertion of the removable needle and the cannula into the user's skin, when the septum housing is pivoted down to the second position alongside the cannula housing portion, this pivoting movement will not pull on the cannula, thereby avoiding inadvertent repositioning or removal of the cannula.

According to one aspect of the invention, an insertion needle assembly is provided, and is removably attachable to the septum housing portion, for insertion of the removable injection needle through the cannula and into the skin. Desirably, the insertion needle assembly is configured so that when the injection needle is in place in the first end of the cannula, subcutaneous injection can be performed with finger pressure, with a speed of injection selected by the user.

In accordance with still another aspect of the invention, a disposable insertion guide housing portion is provided for supporting the cannula housing portion and the insertion handle portion above the injection site prior to injection, allowing the user to preposition the infusion set generally perpendicular to and above the skin surface at the injection site. The infusion set can be pre-packaged with the disposable insertion guide housing portion, ready to use, right off the shelf without needing to be assembled by the user.

In accordance with yet a further aspect of the invention, a needle hub assembly is provided, and also is removably attachable to the septum housing portion. The needle hub assembly includes a needle to penetrate the septum, a device to align the needle with the septum, and a plurality of protective guide rails proximate the needle to protect the user from receiving inadvertent needle sticks.

It is to be understood that both the above general description and the following detailed description are exemplary and explanatory, and are intended to explain the principles of the claimed invention. The accompanying drawings are included to provide a further understanding of the invention and are incorporated and constitute part of the specification, illustrating presently preferred embodiments of the invention.

### BRIEF DESCRIPTION OF THE DRAWINGS

FIG. 1 is a top perspective view of an infusion set in accordance with a first exemplary embodiment of the invention;

FIG. 2 is a side cross-sectional view of along the line 2-2 FIG. 1;

FIG. 3 is a top perspective view of the insertion set of FIG. 1 with the septum housing portion shown separated from the cannula housing portion;

FIG. 4 is an exploded view of the septum housing portion of the first exemplary embodiment of the invention;

FIG. 5 is a side cross-sectional view of the inserted insertion set showing subcutaneous insertion of the cannula and a kink-free bend in the cannula;

FIG. 6 is a perspective view of an exemplary insertion needle assembly useable with the insertion set of FIG. 1;

FIG. 7 is a top perspective view of the exemplary insertion needle assembly of FIG. 6 attached to the exemplary infusion set of FIG. 1;

FIG. 8 is a side cross-sectional view along the line 8-8 in FIG. 7;

FIG. 9 is a front elevation view of the assembly of FIG. 7 with a needle cover attached thereto;

FIG. 10 is a front perspective view of the assembly of FIG. 7 depicting arms of the insertion needle assembly holding side portions of the adhesive pad and paper backing portions folded back to expose adhesive in the center section of the adhesive pad;

FIG. 11 is a front perspective view of the insertion needle assembly of FIG. 6 with a protective cover in place over the insertion needle ready for disposal;

FIG. 12 is a top perspective view of an insertion guide housing supporting the insertion needle assembly of FIG. 6;

FIG. 13 is a side view of the assembly shown in FIG. 12;

FIG. 14 is a top perspective view, similar to FIG. 12, showing the infusion insertion set of FIG. 1 attached to the insertion needle assembly; and

FIG. 15 is a top perspective view, similar to FIG. 14, showing depression of the insertion needle assembly.

FIG. 16 is a bottom perspective view of an exemplary needle hub assembly useable with the infusion set of FIG. 1;

FIG. 17 is a top view of the needle hub assembly of FIG. 16 about to be removably attached to the infusion set of FIG. 1, the septum housing portion being in the second position relative to the cannula housing portion;

FIG. 18 is a top perspective view, similar to FIG. 17, showing attachment of the needle hub assembly to the infusion set of FIG. 1;

FIG. 19 is a side cross-sectional view, similar to FIG. 5, illustrating the needle hub assembly attached to the infusion set of FIG. 1; and

FIG. 20 is a top perspective view, similar to FIG. 18, showing the complete tube extending from the needle hub assembly to a fitting for attachment to an external infusion pump.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

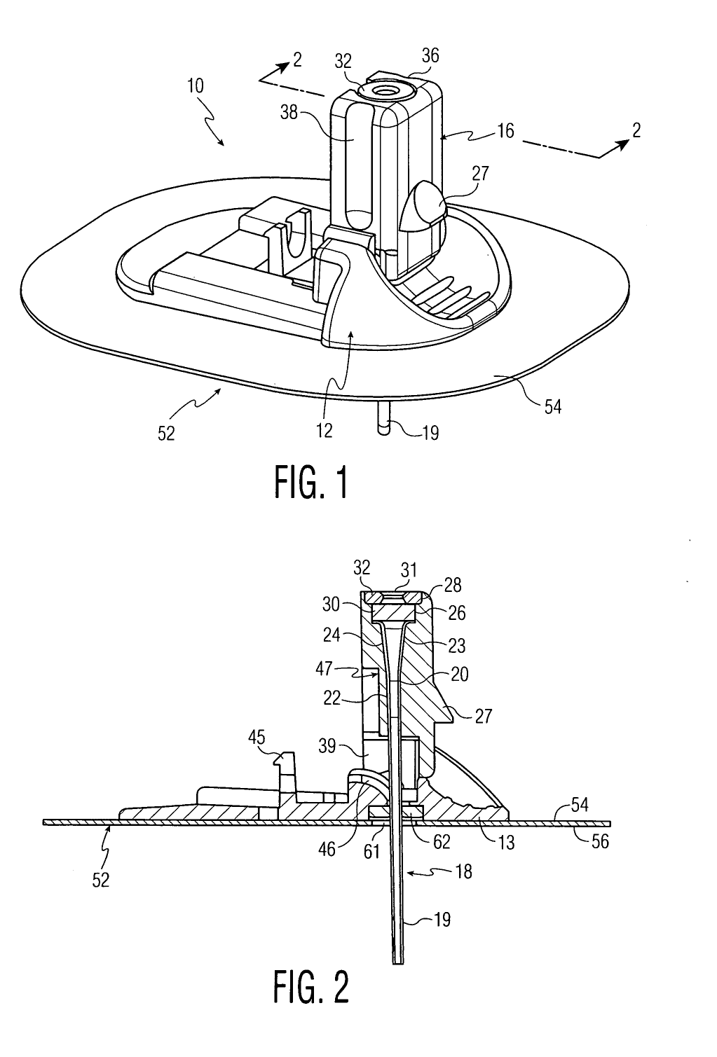

Referring to FIGS. 1-5, infusion set 10 that is a first exemplary embodiment of the present invention is shown. As broadly embodied herein, infusion set 10 includes a multiple-part housing, including first or cannula housing portion 12 and second or septum housing portion 16 pivotably attached thereto. Cannula housing 12 includes base surface 13 configured to provide a stable base for insertion set 10.

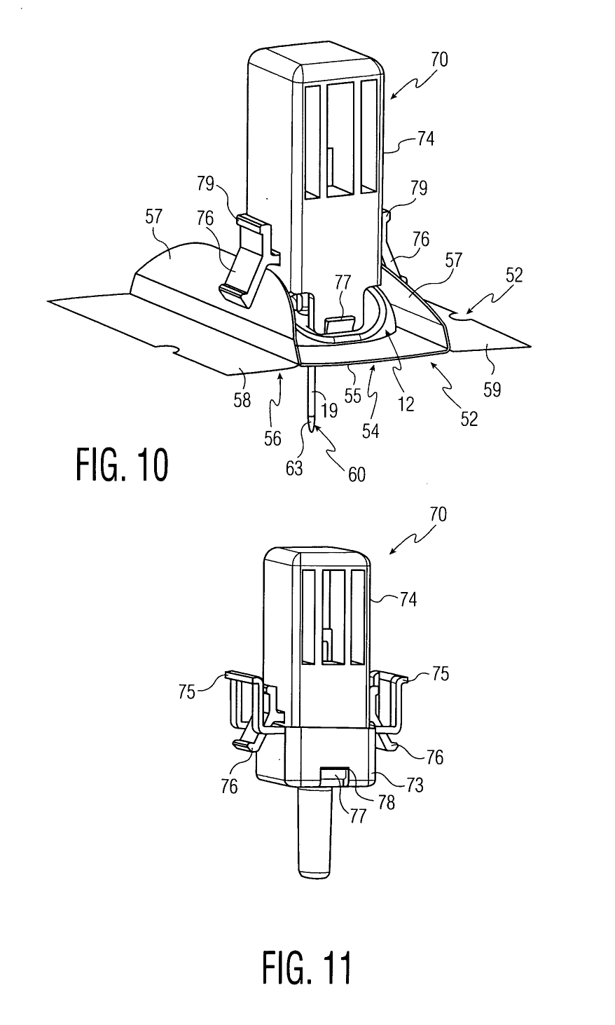

Cannula housing portion 12 is removably attachable to the skin surface 14 of a user via adhesive assembly 52. Adhesive assembly 52 is attached to cannula housing base surface 13 to removably adhere cannula housing portion 12 to skin surface 14. Desirably, adhesive assembly 52 includes sticky adhesive pad 54, which is covered prior to attachment to the skin by a removable paper backing 56. As shown in FIG. 10, pad 54 includes a central portion 55 and two side portions 57. Paper backing 56 includes at least two separately removable paper backing portions 58 and 59. Use of adhesive assembly 52 will be described in more detail hereinafter. Referring again to FIGS. 2-4, adhesive assembly 52 has an aperture 61 defined therethrough for passage of distal end 19 of cannula 18. An annular seal 62 is provided in cannula housing portion 12 proximate aperture 61 to seal aperture 61, and shield the interior of cannula housing portion 12 and the injection site from contamination. Alternatively, annular seal 62 may be omitted and the diameter of the proximate aperture 61 through cannula housing 12 may be made just slightly larger than the outer diameter of cannula 19.

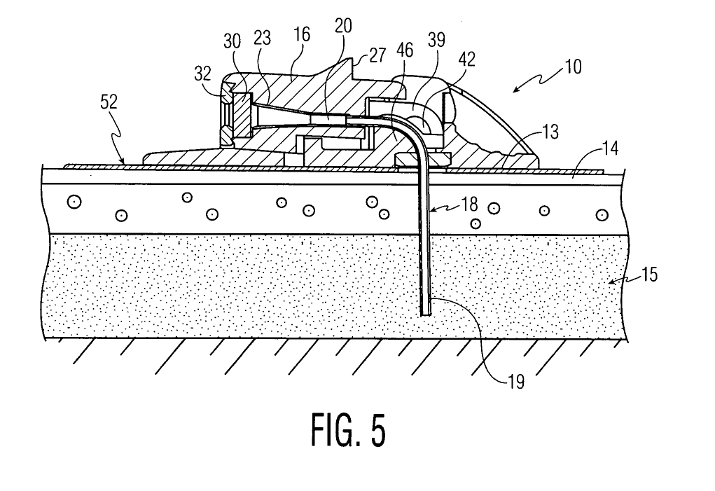

Septum housing portion 16 includes a series of collinear bores 22, 24, 26, and 28, of increasing diameters. Bore 22 has the smallest diameter and extends from a forward end of septum housing portion 16 to bore 24. Bore 24 tapers from bore 24 to bore 26. Bore 26 in turn extends to bore 28. Bore 28 extends to the end of the septum housing portion 16, and has the widest diameter. Bore 24 and a portion of bore 22 are configured to receive ferrule 23. Ferrule 23 has a tapered portion supported in a bore 24 and a cylindrical portion that extends into bore 22. Cannula 18 has a proximal end 20 that is sealingly attached to the cylindrical portion of ferrule 23. Cannula 18 extends out of bore 22 and terminates in distal end 19. As seen in FIG. 2, with septum housing portion 16 connected to cannula housing portion 12, cannula distal end 19 extends through cannula housing portion 12. Cannula 18 desirably is made of medical-grade fluorinated ethylene propylene (FEP) Teflon® or polyurethane, but may be made from any suitable material. As explained in more detail hereinafter, cannula 18 is sized to accept a removable insertion needle to facilitate insertion of distal end 19 of cannula 18 through the user's skin surface 14 and into subcutaneous tissue 15 beneath skin surface 14. (See FIG. 5).

Bore 26 is configured to receive self-sealing elastomeric septum 30 therein. Septum 30 may a round shape as shown, but may be elliptical, rectangular, or any other suitable shape. A stop ring 32 is press fit or the like into bore 28 to retain septum 30 in bore 26. Stop ring 32 has a through bore 31 to permit passage of a needle or the like. Septum 30 seals between stop ring 32 and ferrule 23 to prevent entry of contaminants into cannula 18, however, septum 30 allows passage of a needle or the like. The invention is not limited to the specific configuration of the cannula 18, ferrule 23, septum 30 and stop ring 32 illustrated herein. Other configurations of supporting cannula 18 extending from septum housing portion 16 may also be used.

Septum housing portion 16 includes vertical alignment grooves 36 and 38 defined in the sides thereof, and a pair of depending legs 39 and 40. Each of the depending legs 39 and 40, includes a respective aperture 42 and 44. Apertures 42 and 44 are sized to snap-fit over corresponding pins 35, 37, respectively, projecting from cannula housing portion 12. Engagement of pins 35, 37 and apertures 42, 44 defines a hinged connection for pivotal movement of septum housing portion 16 relative to cannula housing portion 12. Alternately, pins can be provided on septum housing portion 16 with corresponding apertures in cannula housing portion 12. Other forms of pivotal mounting of septum housing 16 relative to cannula housing portion 12 may also be utilized.

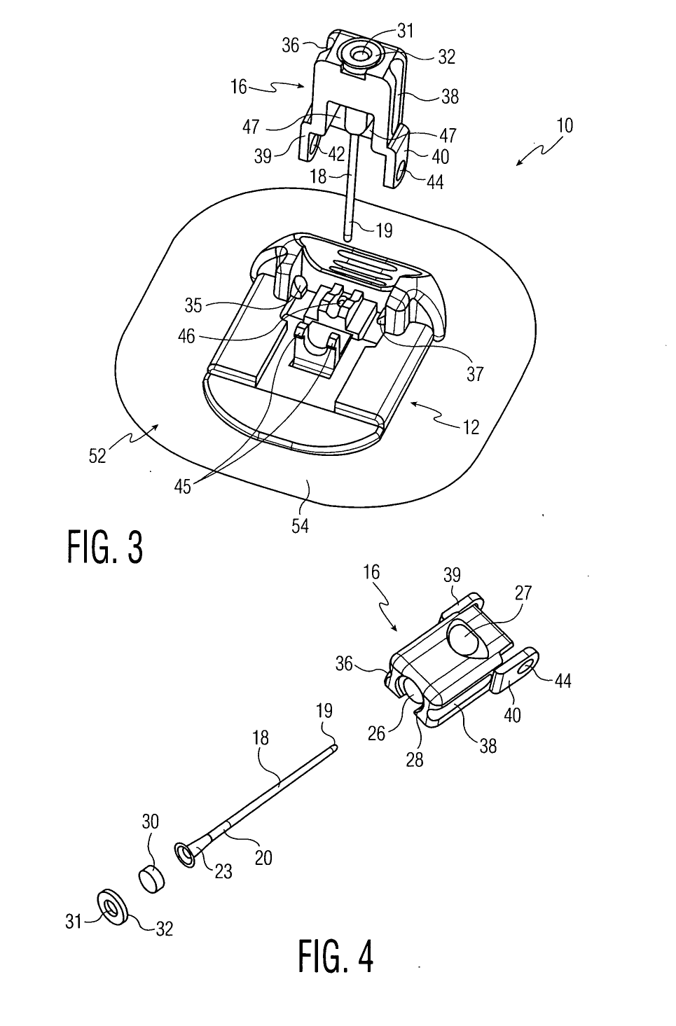

Septum housing portion 16 is configured to pivot with respect to cannula housing portion 12 between a first position as shown in FIGS. 1 and 2 and a second position as shown in FIG. 5. In the first position, the axis of septum housing portion 16 extends substantially perpendicular to cannula housing portion base 13, and thereby, substantially normal to skin surface 14 to facilitate a straight insertion. After insertion of cannula 18, septum housing portion 16 is pivoted to the second position where the axis of septum housing portion 16 is substantially parallel to skin surface 14. In the second position, insertion set 10 provides a low profile relative to skin surface 14. As shown in FIGS. 2 and 3, a pair of flexible latching arms 45 extend from cannula housing portion 12 and are configured to mate with latching depressions 47 on septum housing portion 16 to lock septum housing portion 16 in place relative to the cannula housing portion 12 in the second position.

The point of pivotal attachment between septum housing portion 16 and cannula housing portion 12 is selected according to a geometrical calculation, such that when septum housing portion 16 pivots relative to cannula housing portion 12, cannula 18 is neither pulled nor pushed. In this way, cannula 18 will not be repositioned with respect to skin surface 14, and will not be inadvertently pulled out of the skin when septum housing portion 16 is pivoted. A geometric calculation of a pivot offset for an axis of rotation by vertical cannula 18 (cannula 18 is vertical when the septum housing portion 16 is in the first position), is shown below:

[image]

To further protect cannula 18, cannula housing portion 12 further includes a cannula guide or curved mandrel 46, positioned to support an intermediate portion of cannula 18 proximate skin surface 14. Cannula guide 46 provides an arcuate path for cannula 18. As septum housing portion 16 is pivoted to the second position, cannula guide 46 supports the intermediate portion of cannula 18 along a gradual curve. The arcuate path is approximately a fraction of a 90° bend, with a radius in the range of approximately 1-4 mm and desirably about 2.25 mm. This slight curve prevents kinking of cannula 18.

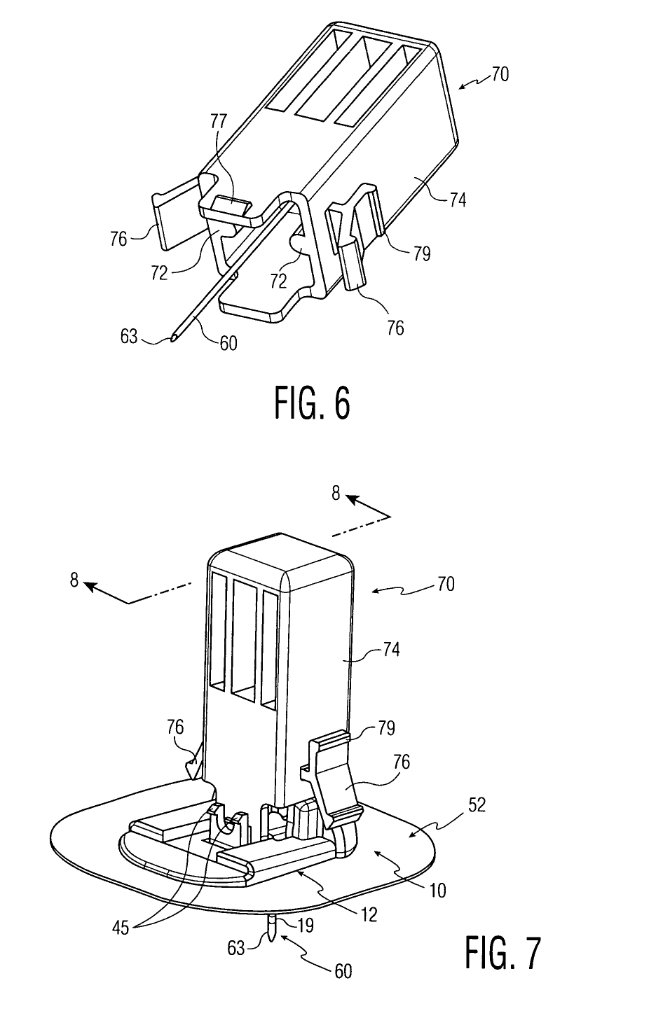

Various insertion needles can be removably inserted through the cannula 18 to insert distal end 19 of cannula 18 into the skin surface 14. A first embodiment of an insertion needle assembly 70 will be described reference to FIGS. 6-11. The invention is not limited to insertion needle assembly 70 and the insertion set 11 may be used with various insertion needles.

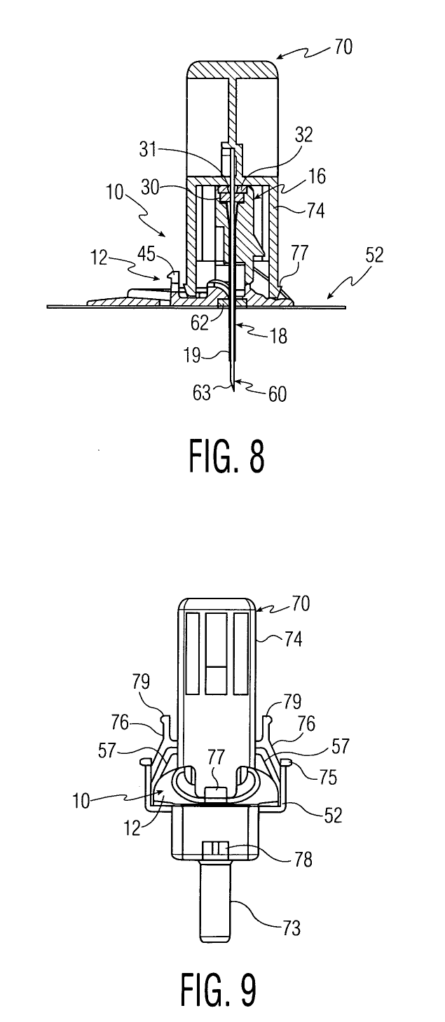

Insertion needle assembly 70 includes a generally hollow housing 74 configured to removably attach to the septum housing portion 16. Removable insertion needle 60 extends from the interior of housing 74 of insertion needle assembly 70. Insertion needle 60 can have various configurations, including but not limited to, a standard beveled needle or a trocar having a sharp tip 63. Internal guide rails 72 on housing 74 are configured to fit slidably into vertical alignment grooves 36, 38 on the sides of septum housing portion 16. Interaction between guide rails 72 and alignment grooves 36, 38 aligns the needle 60 with stop ring throughbore 31 such that insertion needle 60 penetrates septum 30 and passes through cannula 18 out cannula distal end 19 as shown in FIG. 8. Friction between insertion needle 60 and septum 30 and/or cannula 18 is generally high enough to hold insertion needle assembly 70 to insertion set 10 without an additional locking mechanism. A lock mechanism can be provided if desired. Guide rails 72 and alignment guides 36, 38 prevent insertion needle assembly 70 from rotating relative to septum housing portion 16.

Referring to FIG. 9, infusion set 10 may be supplied prepackaged with insertion needle assembly 70 attached thereto. To protect users and maintain the sterility of the prepackaged assembly, cover 73 may be positioned over needle 60 and attached to insertion needle assembly 70. Cover 73 includes extension brackets 75 configured to removably engage extension arms 76 extending from the sides of insertion needle assembly housing 74. The extension brackets 75 also act to fold up side portions 57 of attachment assembly 52, thereby reducing the shipping size of the prepackaged assembly and protecting attachment assembly 52.

To prepare infusion set 10 for use, cover 73 is removed from insertion needle assembly 70, thereby exposing cannula 18 and insertion needle 60. Upon removal of cover 73, extension arms 76 of insertion needle assembly 70 continue to maintain the side portions 57 of the adhesive pad 54 from drooping down as shown in FIG. 10. Prior to insertion, the two halves 58, 59 of backing paper 56 are folded away from center portion 55 of adhesive pad 54 to expose the adhesive surface. Backing paper 56 remains attached to side portions 57 of pad 54 which remain retained by extension arms 76. This allows a wide mounting pad 54 to be used without the side portion 57 from dropping and contacting the skin, as often occurs with prior art devices.

To insert insertion set 10, a user holds insertion needle assembly housing 74 in one hand while pinching a fold of skin with the other hand. Septum housing portion 16, and thereby insertion needle 60, are perpendicular to skin surface 14. Insertion needle assembly housing 74 is pressed straight downward by the user against the user's skin to thereby insert insertion needle 60 and distal end 19 of cannula 18 through skin surface 14 in an orientation substantially normal to the skin surface 14. The force required to insert needle 60 is generally less than 0.5 pounds, and needle 60 desirably is inserted into the skin using only finger pressure, with the speed of insertion being controlled by the user.

Needle 60 is inserted until center portion 55 of adhesive pad 54 contacts skin surface 14. Side portions 57 of pad 54 are applied to skin surface 54 by pulling the halves 58, 59 of backing paper 56 parallel to skin surface 14 while pressing side portions 57 against skin surface 14. Needle 60 is removed by drawing insertion needle assembly 70 away from insertion set 10. Cover 73 can then be positioned over needle 60, as illustrated in FIG. 11, for disposal of insertion needle assembly 70. To maintain cover 73 on insertion needle assembly 70, protrusions 77 (shown in FIGS. 8-15) extending from insertion needle assembly housing 74 engage notches 78 in cover 73 (shown in FIG. 11). Once needle 60 is removed, the user pivots septum housing portion 16 down to the second position, and locks it in place as shown in FIG. 5. By engaging 45 into 47, as explained above, curved mandrel 46 prevents kinking of cannula 18. Insertion set 10 is ready for connection to a needle hub assembly 90 as will be described hereinafter.

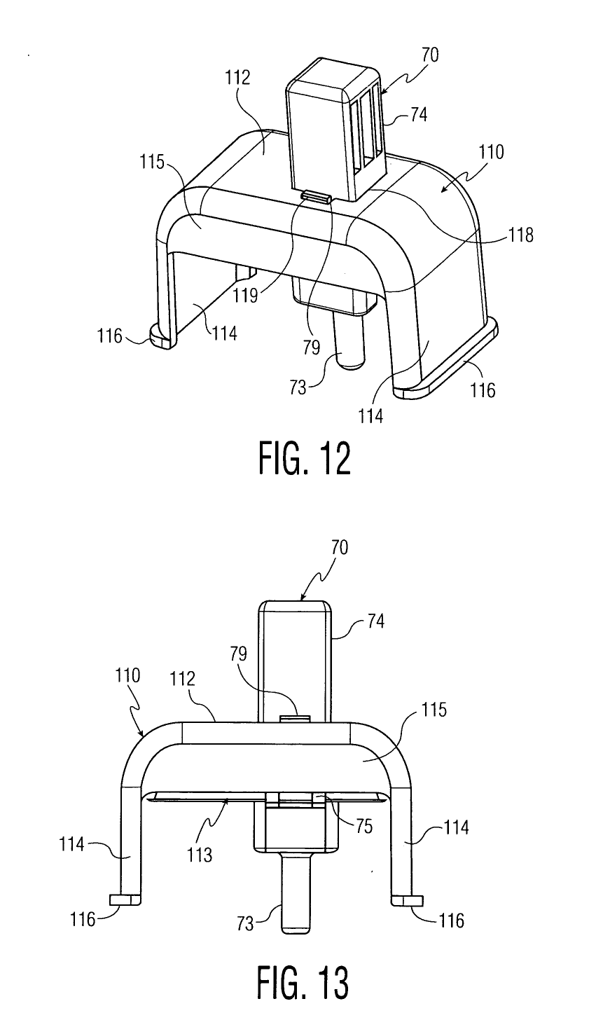

To assist in the insertion process, disposable insertion guide housing 110, as illustrated in FIGS. 12-15, may be utilized with insertion needle assembly 70. Insertion guide housing 110 has a generally U-shaped configuration with opposed legs 114 extending downwardly from top surface 112. Each leg 114 terminates in a contact foot 116. Insertion guide housing 110 is configured such that top surface 112 is held generally parallel to skin surface 14 by contact feet 116. Finger grasp sides 115 extend between legs 114 along each side of guide housing 110.

Opening 118 is defined in top surface 112 and has a cross-sectional shape that generally complements the shape of insertion needle assembly housing 74 such that the preassembled insertion set 10 and insertion needle assembly 70, as shown in FIG. 9, may be positioned into insertion guide housing 110. Opening 118 includes a pair of opposed notches 119 configured to receive upper projections 79 extending from the insertion needle assembly extension arms 76 to retain insertion needle assembly 70 extending through insertion guide housing top surface 112, as shown in FIGS. 12 and 13. The assembly may be preassembled and ready for use as illustrated in FIGS. 12 and 13.

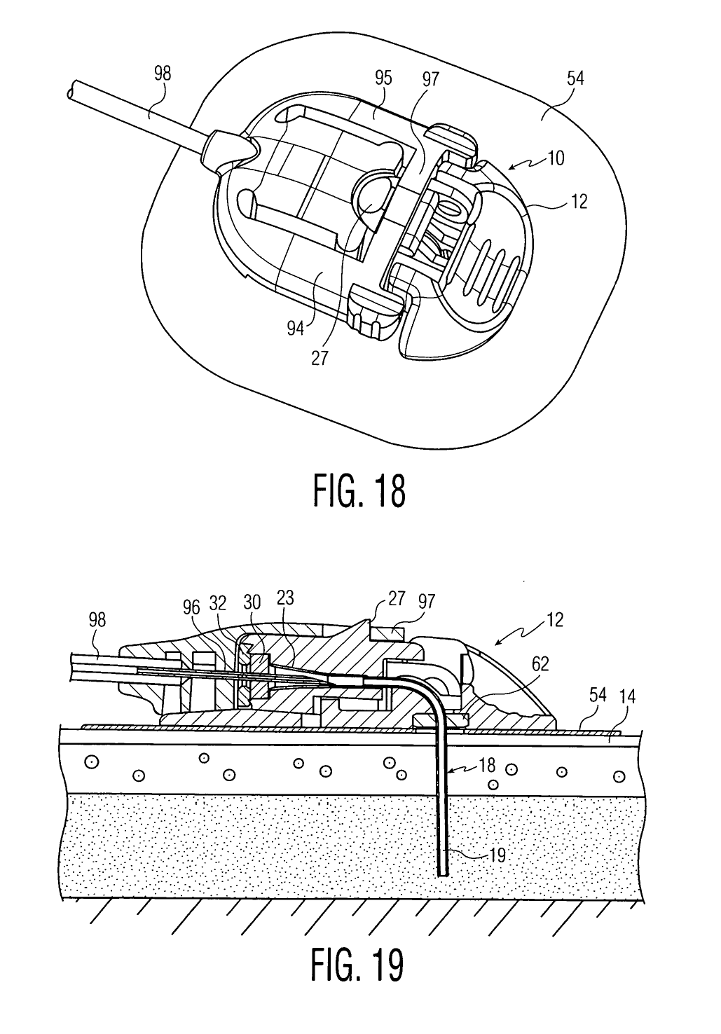

Insertion guide housing 110 has generally open sides 113 which permit flaps of the backing paper 56 on adhesive pad 54 to be folded up parallel to the cannula housing portion bottom while adhesive pad 54 is being adhered to the skin, while the cannula housing portion 12 is mounted in the insertion guide housing 110, as shown in FIG. 18.

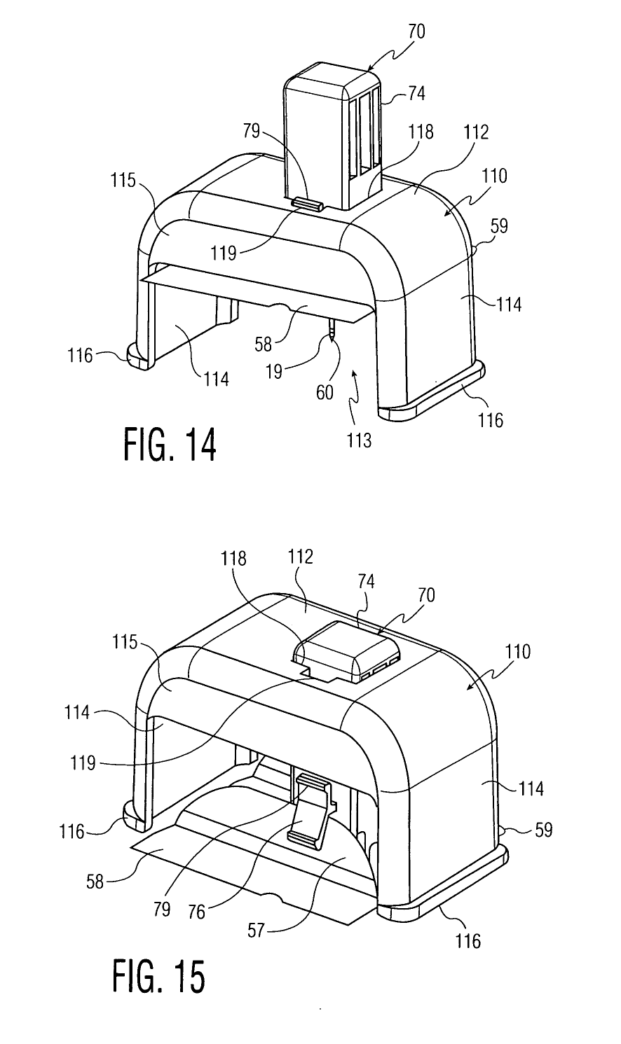

To operate the system, the user picks up the insertion guide housing 110 with one hand using the opposed finger grasp sides 115. As supplied, insertion needle 60 extends through cannula 18, with adhesive pad side portions 57 tucked under the arms 76 of the insertion needle assembly 70, similar to that shown in FIGS. 9 and 10. The user removes needle cover 73 and folds back both portions 58 and 59 of backing paper 56 to expose central portion 55 of pad 54, as shown in FIG. 14. Backing paper portions 58 and 59 may be folded up along the finger grasp sides 115 and held out of the way by the user. The user presses feet 116 of insertion guide housing 110 against the skin at the injection site. The user then pushes downward on the top of housing 74 of insertion needle assembly 70 with a light finger force of about 0.1-0.5 lbf. Projections 79 flex and insertion needle assembly 70 pushes needle 60 along with insertion set 10 downward toward skin surface 14. Needle 60 is inserted through the skin surface 14 and cannula distal end 19 moves through aperture 61 to penetrate the skin surface 14 subcutaneously in an orientation substantially normal to skin surface 14. Needle penetration of the skin with finger pressure typically is less painful than spring-loaded needle penetration. The user then pulls away the folded backing paper 56 on each side, applying the side portions of the adhesive pad to the skin without wrinkling pad 54. The user then removes insertion guide housing 110 and insertion needle assembly 70 from septum housing portion 16, thereby withdrawing insertion needle 60 while leaving cannula 18 in place. Septum housing portion 16 is pivoted and locked in the second position illustrated in FIG. 5. Protective cover 73 is placed over needle 60 and insertion guide housing 110 and insertion needle assembly 70 can be discarded.

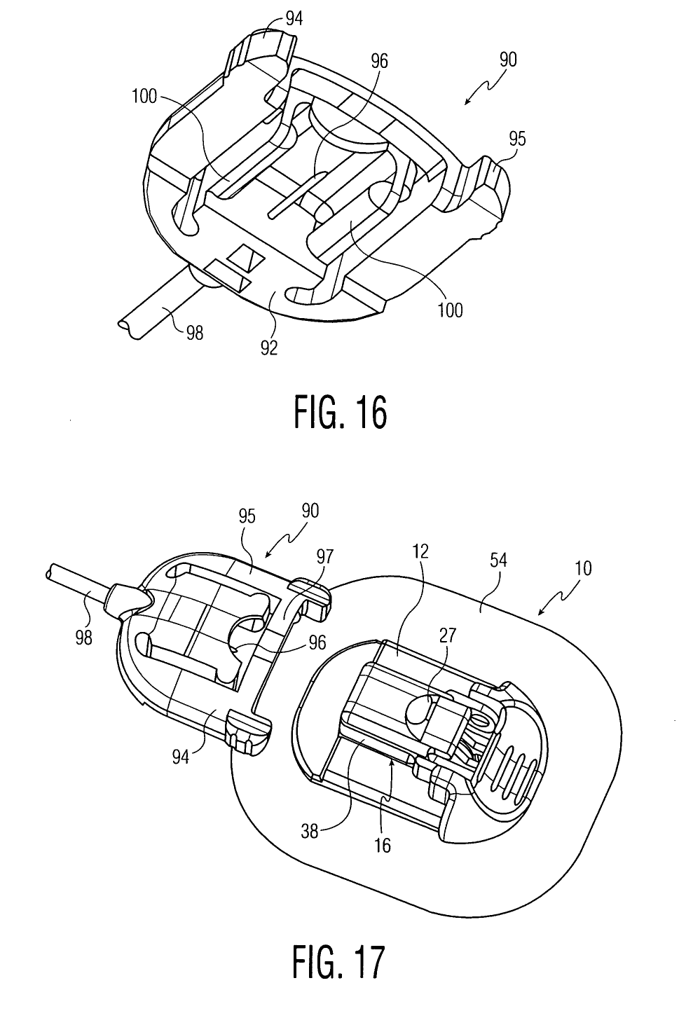

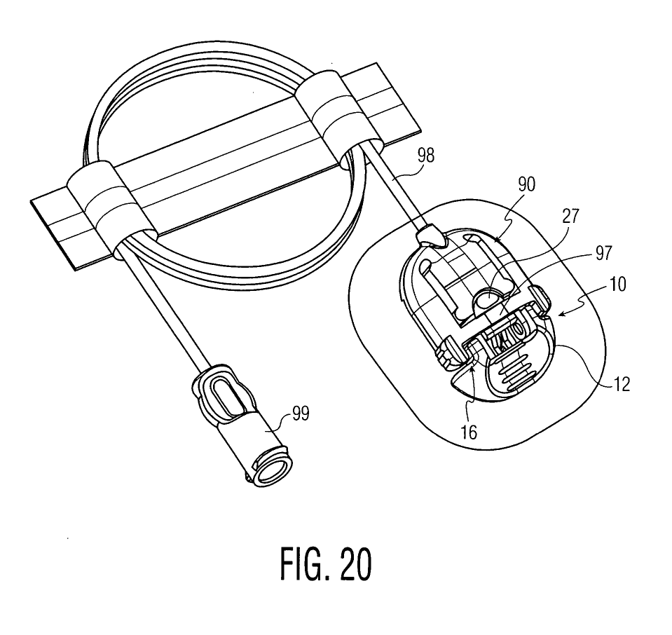

Once insertion set 10 is positioned on the user, in one of the manners above or any other suitable manner, insertion set 10 is ready for connection to an infusion source, for example, an infusion pump, through a needle hub assembly. A first exemplary embodiment of a needle hub assembly 90 useable with insertion set 10 is illustrated in FIGS. 16-20. Needle hub assembly 90 is configured to be removably attached to insertion set 10.

Needle hub assembly 90 includes a substantially flat housing 92, resilient band 97, flexible arms 94 and 95, and needle 96. Needle hub assembly 90 also includes guide rails 100 extending along a lower surface of housing 92, inward of flexible arms 94 and 95. Guide rails 100 are configured to align with and slide into grooves 36 and 38 in the sides of septum housing 16. Needle 96 projects between guide rails 100 below an upper surface of housing 92 such that needle 96 is substantially enclosed therein and the user is protected from inadvertent needle sticks when needle hub assembly 90 is disconnected from septum housing portion 16. Sliding of guide rails 100 into respective grooves 36 and 38 guides needle 96 through stop ring 32 and septum 30 such that needle 96 terminates in ferrule 23 as shown in FIG. 19.

To retain needle hub assembly 90 in engagement with septum housing portion 16, resilient band 97 extends between flexible arms 94 and 95 and is configured to flex over and be retained by a retaining bump 27 on septum housing portion 16 as shown in FIG. 18. Retaining bump 27 desirably has a ramped profile when viewed from the side (see FIG. 2) such that resilient band 99 slides freely over retaining bump 27 as needle housing assembly 90 is connected to septum housing portion 16. Resilient band 99 may make an audible click to give the user positive acknowledgment that needle hub assembly 90 and septum housing portion 16 are locked together. To remove needle hub assembly 90, arms 94 and 95 are squeezed together such that resilient band 99 flexes upward to a central height greater than the height of retaining bump 27. Needle hub assembly 90 is slid off of septum housing portion 16, thereby removing needle 96 from septum 30. Other types of engaging and locking assemblies may also be used. For example, a set of complementary barbs and notches (not shown) can be provided on opposing surfaces of needle hub assembly 90 and septum housing portion 16. Other assemblies may also be utilized.

Flexible tube 98 is attached to needle 96 and projects from a rear end of needle hub assembly 90. Tube 98 can extend directly to a medication source, such as an insulin pump, or to an appropriate fitting 99, such as a Luer fitting, which can be connected to an external infusion pump (not shown) or another medicine source. Operation of the medication source supplies medicine through tube 98, through needle 96 and through cannula 18 to deliver the of medicine to the user.

It will be apparent to those skilled in the art that various modifications and variations can be made in the infusion set of the present invention, without departing from the spirit or scope of the invention. Thus, the present invention covers modification and variations of the invention, provided they fall within the scope of the claims, and their equivalents.

What is claimed:

1. An infusion set comprising:
a cannula housing portion having a base surface;
a septum housing portion adapted to be pivotally attached to said cannula housing portion, said septum housing portion having an axis and being pivotable between a first position wherein said axis is substantially perpendicular to said base surface and a second position wherein said axis is substantially parallel to said base surface; and
a cannula supported in and extending from said septum housing portion such that said cannula is substantially parallel to said axis when said septum housing portion is in the first position.

2. The infusion set of claim 1 wherein said cannula housing portion includes a cannula guide configured to support a portion of said cannula along an arcuate path when said septum housing portion is in the second position.

3. The infusion set of claim 2 wherein said arcuate path comprises at least a fraction of a 90° turn.

4. The infusion set of claim 1 wherein a septum is positioned in said septum housing portion to seal a needle opening to said cannula.

5. The infusion set of claim 1 further comprising an adhesive assembly attached to said base surface.

6. The infusion set of claim 5 wherein said adhesive assembly comprises an adhesive pad and a removable backing.

7. The infusion set of claim 6 wherein portions of said backing are separately removable from said adhesive pad.

8. The infusion set of claim 6 wherein said adhesive pad defines an aperture therein for passage of said cannula therethrough.

9. The infusion set of claim 8 wherein said cannula housing portion includes an annular seal about said cannula adjacent said aperture.

10. The infusion set of claim 1 wherein said septum housing portion is pivotally attached to said cannula housing at a selected pivot point.

11. The infusion set of claim 10 wherein said pivot point is selected so that pivoting of said septum housing portion relative to said cannula is independent of the position of the cannula.

12. The infusion set of claim 1 further comprising a latching mechanism for fixing said septum housing portion to said cannula housing portion when said septum housing portion is in said second position.

13. The infusion set according to claim 1 further comprising an insertion needle assembly including an insertion needle assembly housing and a needle supported thereby, wherein said insertion needle assembly housing is configured to engage the septum housing portion with said needle axially aligned with said cannula such that said needle is removably positionable through said cannula.

14. The infusion set of claim 13 wherein said insertion set further comprises an adhesive assembly including an adhesive pad and a backing material, said insertion needle assembly further comprising an arm for holding a portion of the backing material during insertion.

15. The infusion set of claim 13 wherein the insertion needle assembly includes at least one guide rail adjacent the needle and configured to slidably engage a corresponding groove in said septum housing portion.

16. The infusion set of claim 13 further comprising an insertion guide housing having a u-shaped configuration defining a top surface and a pair of legs depending therefrom, the top surface having an opening having a configuration which complements an external configuration of said insertion needle assembly housing.

17. The infusion set of claim 16 wherein the insertion needle assembly housing includes flexible projections configured to engage the insertion guide housing adjacent the opening to support the insertion set in a position for insertion.

18. The infusion set according to claim 1 further comprising a needle hub assembly removably attachable to said septum housing portion and connectable to a source of medication.

19. The infusion set of claim 18 wherein said needle hub assembly includes an infusion needle positionable within the cannula.

20. The infusion set of claim 19 wherein the needle hub assembly includes at least one guide rail adjacent the infusion needle and configured to slidably engage a corresponding groove in said septum housing portion.

21. A method of inserting a cannula through skin of a user, comprising:
providing an infusion set comprising:
a cannula housing portion having a base surface;
a septum housing portion pivotally attached to said cannula housing portion, said septum housing portion having an axis and being pivotable between a first position wherein said axis is substantially perpendicular to said base surface and a second position wherein said axis is substantially parallel to said base surface; and
a cannula supported in and extending from said septum housing portion such that said cannula is substantially parallel to said axis when said septum housing portion is in the first position;
positioning said septum housing portion in the first position;
removably positioning an insertion needle through said cannula;
inserting said insertion needle through said skin such that said cannula, at the injection site, is oriented substantially normal to said skin;
removing said insertion needle from said cannula; and
pivoting said septum housing portion to the second position.

22. The method of claim 21 further comprising attaching said septum housing portion pivotally to said cannula housing at a selected pivot point.

23. The method of claim 22 wherein said pivot point is selected so that pivoting of said septum housing portion relative to said cannula is independent of the position of the cannula.

24. The method of claim 21 further comprising latching said septum housing portion to said cannula housing portion when said septum housing portion is in said second position.

25. The method of claim 21 wherein removably positioning a needle through said cannula comprises:
providing an insertion needle assembly including an insertion needle assembly housing that supports the needle; and
engaging the septum housing portion with said insertion needle assembly housing such that said insertion needle is removably positioned through said cannula.

26. The method of claim 25 wherein said insertion set further comprises an adhesive assembly including an adhesive pad and a backing material and said insertion needle assembly further comprises at least one arm, the method further comprising holding a portion of the backing material with the at least one arm during insertion.

27. The method of claim 25 further comprising:
providing an insertion guide housing having a substantially u-shaped configuration defining a top surface and a pair of legs depending therefrom, the top surface including an opening having a configuration which complements an external configuration of said insertion needle assembly housing; and
supporting the insertion needle assembly housing in the opening.

28. The method of claim 21 further comprising:
attaching a needle hub assembly to said septum housing portion; and
connecting said needle hub assembly to a source of medication.

29. An infusion set comprising:
a first housing portion;
a second housing portion pivotally attached to said first housing portion;
a cannula supported by said second housing portion and extending through an aperture in the first housing portion; and
a cannula guide in said first housing portion adjacent the aperture and configured to support a portion of said cannula along an arcuate path.

30. An infusion assembly comprising:
a first housing portion having a base surface;
a second housing portion having an axis and being pivotally attached to said first housing portion for pivotal movement between a first position wherein said axis is perpendicular to said base surface and a second position wherein said axis is substantially parallel to said base surface;
a cannula supported by said second housing portion such that the cannula is parallel to said axis when said second housing portion is in the first position; and
an insertion needle assembly engageable with the second housing portion when the second housing portion is in the first position, the insertion needle assembly including an insertion needle removably positioned through the cannula when said second housing portion is in the first position.
